(19) United States
(12) Patent Application Publication
(10) Pub. No.: US 2024/0049963 A1
(43) Pub. Date: Feb. 15, 2024

(54) Cover-Uncover Test in a VR/AR Headset

(71) Applicant: Twenty Twenty Therapeutics LLC, South San Francisco, CA (US)

(72) Inventors: Supriyo Sinha, Menlo Park, CA (US); Kirk Gossage, Pacifica, CA (US); Dimitri Azar, Chicago, IL (US)

(21) Appl. No.: 18/447,936

(22) Filed: Aug. 10, 2023

Related U.S. Application Data
(60) Provisional application No. 63/397,112, filed on Aug. 11, 2022.

(51) Int. Cl. A61B 3/113; G06T 7/73; G09G 3/00; A61B 3/00

(52) U.S. Cl. CPC A61B 3/113; G06T 7/73; G09G 3/001; A61B 3/0058; A61B 3/005; G06T 2207/30201; G09G 2354/00; G09G 2380/08

(57) ABSTRACT

Virtual reality, VR, headset-based and augmented reality, AR, headset-based electronic systems that can be used to perform cover/uncover tests. The systems may improve the sensitivity, consistency, and ease of application of the cover/uncover test. Other aspects are also described.

### CROSS-REFERENCE TO RELATED APPLICATION

This nonprovisional patent application claims the benefit of the earlier filing date of U.S. Provisional Application No. 63/397,112 filed Aug. 11, 2022.

### FIELD

An aspect of the disclosure here relates to equipment that can be used for performing cover-uncover eye testing.

### BACKGROUND

Traditionally, cover/uncover tests are performed by an eye-care professional (ECP) who holds an opaque or translucent occluder over one of a person's eyes while asking the person to fixate on a point. The ECP observes the movement of the uncovered eye, while the opposite eye is covered, and then tries to observe the movement of the covered eye just after it is uncovered. The ECP then notes their observations.

### SUMMARY

Traditional cover/uncover tests suffer from two problems. First, the ECP is unable to observe the position of the person's eye while that eye is covered. Second, the observations of eye movement made by the ECP do not have sufficient resolution or detail to be useful for quantifying a condition of the eye and tracking progression of the condition over time.

One aspect of the disclosure here is a virtual reality, VR, headset-based electronic system that can be used to perform cover/uncover tests. Another aspect is an augmented reality, AR, headset-based system that can also be used to perform cover/uncover tests. Any reference here to a VR headset or an AR headset is understood to encompass a mixed reality headset that may have aspects of both a VR headset and an AR headset that are relevant to the disclosure here. These systems may improve the sensitivity, consistency, and ease of application of the cover/uncover test; the test is conventionally performed manually by a trained ECP, with results or sensitivity that vary by practitioner. The results of the cover/uncover test may then be used to diagnose strabismus and/or amblyopia.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have advantages that are not recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is a diagram of an example virtual reality, VR, headset-based system that is suitable for cover/uncover testing.

FIG. 2 depicts how the eyes of a user should appear when the user who has no tropia (but may or may not have a phoria) is staring at a single fixation point.

FIG. 3 depicts how the eyes of the user with no tropia would appear when one of their eyes is covered.

FIG. 4 depicts how the eyes of a user who has exotropia in the left eye would appear.

FIG. 5 depicts how the eyes of the user who has exotropia in the left eye would appear when the left eye is covered.

FIG. 6 depicts how the eyes of the user who has exotropia in the left eye would appear when the right eye is covered.

FIG. 7 depicts the eyes of a user who has esophoria, as they would appear when their right eye is covered (once the fusion between their eyes has been broken).

FIG. 8 shows graphs of the tracked x-position (horizontal) and tracked y-position (vertical) of left and right eyes of a user during cover/uncover testing.

FIG. 9 is a diagram of an example augmented reality, AR, headset-based system for cover/uncover testing.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

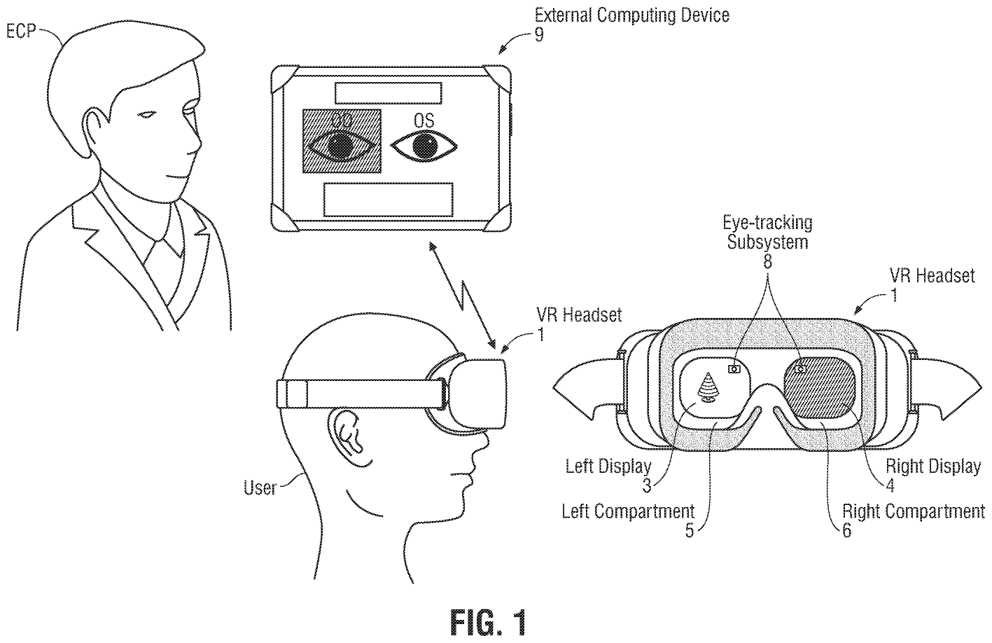

FIG. 1 is a diagram of an example virtual reality, VR, headset-based system that can be used for cover/uncover testing. The system is composed of a VR headset 1 (or a mixed reality headset having relevant features of a VR headset), which has a wired or wireless communication network interface for communicating data with an external computing device 9, e.g., a tablet computer, a laptop computer, etc. A person, such as an eye care professional, ECP, interacts with software that is being executed by one or more microelectronic data processors (generically, “a processor”) of the system. The software may have components that are being executed by a processor that is in the VR headset 1, and it may have components that are executed by a processor which is part of the external computing device 0, while the VR headset 1 is fitted over the user's eyes. Some of these software components may be executed either in the VR headset 1 or in the external computing device 9.

The VR headset 1 is composed of a left visible light display 3 to which a left compartment 5 is coupled that fits over the left eye of the user, and a right visible light display 4 to which a right compartment 6 is coupled that fits over the right eye of the user. The left and right compartments are configured, e.g., shaped and being opaque, so that the user cannot see the right display 4 using only their left eye, and the user cannot see the left display 3 using only their right eye (once the VR headset 1 has been fitted over the user's eyes). Also, the left and right displays need not be separate display screens, as they could instead be the left and right halves of a single display screen. The displays may be implemented using technology that provides sufficient display resolution or pixel density, e.g., liquid crystal display technology, organic light emitting diode technology, etc. Although not shown, there may also be an eyecup over each of the left and right displays that includes optics (e.g., a lens) serving to give the user the illusion that the object they see in the display (in this example a pine tree, which may be displayed in 2D or in 3D) is at a greater distance than the actual distance from their eye to the display, thereby enabling more comfortable viewing. The headset might also incorporate trial lenses or some other adjustable refractive optical system to accommodate patients with different refractive errors. The VR headset 1 also has a non-visible light-based eye tracking subsystem 8, e.g., an infrared pupil tracking subsystem, for (invisible to the user) independently tracking the positions of the left and right eyes (as well as blinks and pupil size).

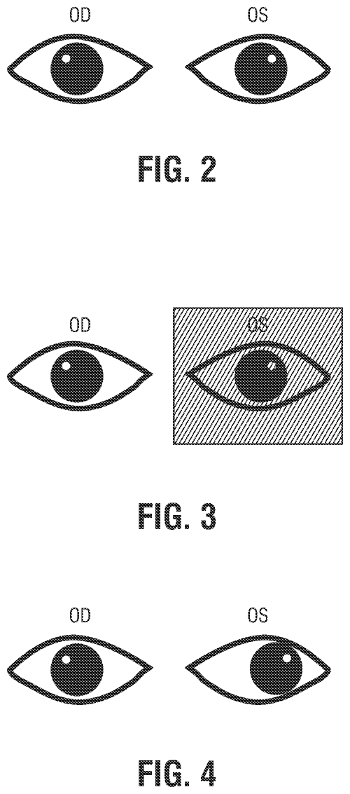

The system has a processor (e.g., one or processors that are part of the external computing device 9, one or more that are within the housing of the VR headset 1, or a combination of processors in those devices that are communicating with each other through a communication network interface) that is configured by software to conduct a cover/uncover test, when the headset has been fitted over the user's eyes. To do so, the processor signals the left and right visible light displays to display an object that the user sees as a single object using their left and right eyes simultaneously. In the example shown, the object is a pine tree but more generally it can be any graphical object that serves as a fixation point at which the user is asked to stare. The fixation point is provided to both eyes in the same angular space, so that it appears as a single object if the user's eyes are aligned, e.g., the user is one who has no tropia. Such a user's eyes would appear as depicted in FIG. 2.

Next, the processor records the tracked position of the right eye (using output data from the eye tracking subsystem 8) while signaling the right display 4 to stop displaying the object, while the object remains displayed in the left display 3—this is depicted in FIG. 1 where the right display 4 is shown as shaded and the pine tree is no longer visible in it. In this state, the right eye position is being tracked and recorded even when there is no visible light illuminating the right eye (because the right display 4 has been darkened) such that the right eye is said to be “covered”, while the left display remains illuminated and displaying the object and so the left eye is “uncovered” (the object remains visible and preferably stationary in the left display 3). For that state, if the user has no tropia, then their eyes would appear as depicted in FIG. 3, namely aligned. Note the shading over the right eye representing that the right eye is covered.

The processor may be configured to selectively “cover” (in the manner described above) the left eye only, and the right eye only, at different times during the cover/uncover test, while recording the tracked position of at least the covered eye (and perhaps also the uncovered eye). In other words, the processor may record the tracked position of the right eye (using output from the eye tracking subsystem 8), while signaling the right visible light display 4 to stop displaying the object while the object remains displayed in the left display 3. In that state, the processor signals the right visible light display 4 so the object disappears from the right visible light display 4 but remains displayed as stationary in the left visible light display 3. As an example, the right display 4 is signaled to darken until the object disappears, e.g., to darken completely (to produce no visible light). In that state, the user's right eye would see complete darkness since the right compartment 6 is entirely opaque and optically seals off the right eye. Similarly, the left compartment 5 may be entirely opaque and it optically seals off the left eye so that the user sees complete darkness through their left eye whenever the left visible light display 3 has been darkened completely.

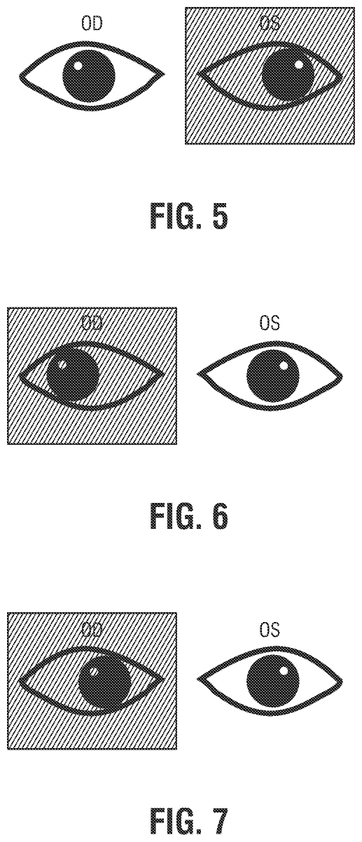

Turning now to FIG. 3, this figure depicts how the eyes of the user with no tropia would appear when one of their eyes is covered, e.g., the eyes are aligned or centered—there is no misalignment of either eye. In contrast, FIG. 4 depicts how the eyes of a user who has exotropia in the left eye would appear. If the user's left eye were to then be covered as shown in FIG. 5, there will be no movement of the eyes (as shown). But if the right eye were to be covered instead, the left eye will turn inward to allow the left eye to focus on the fixation point as shown in FIG. 6. And since the left and right eyes are yoked (a motor coordination that drives movement of both eyes in the same direction to maintain binocular gaze), the right eye will turn outward as shown in FIG. 6. The processor directs such cover/uncover testing by signaling the left and right and displays in sequence (or at different times) to “cover” and “uncover” as follows: signal only the left display to stop displaying the object, e.g., go completely dark, and then signal the left display to resume displaying the object, and then signal the right display to stop displaying the object, e.g., go completely dark, and then signal the right display to resume displaying the object. During this entire sequence (e.g., as each of the left and right displays transitions into its completely dark state and goes back to displaying the object, one at a time), the processor is also recording the tracked positions of both eyes, using output from the eye tracking subsystem.

The systems described here may also be used to perform cover/uncover testing whose results can also be used to detect phorias. In contrast to tropia which is a physical misalignment in one or both eyes, a phoria is a deviation of one eye that may only be present when the eyes are not looking at the same object. To produce cover/uncover test results that can be used to detect phorias, the processor may be configured to repeatedly switch the “cover” from the left eye to the right eye and back again (as one or more repetitions), which may result in the brain's fusion between the eyes being broken or suspended. FIG. 7 depicts the eyes of a user who has esophoria, as they would appear when their right eye is covered (once the fusion between their eyes has been broken). Meanwhile, the processor is recording the tracked positions of the left eye and the right eye during this sequence (while signaling the left display to cover the left eye and then signaling the right display to cover the right eye while signaling the left display to uncover the left eye) and records the movement of the right eye when the right eye becomes covered (as shown in FIG. 7).

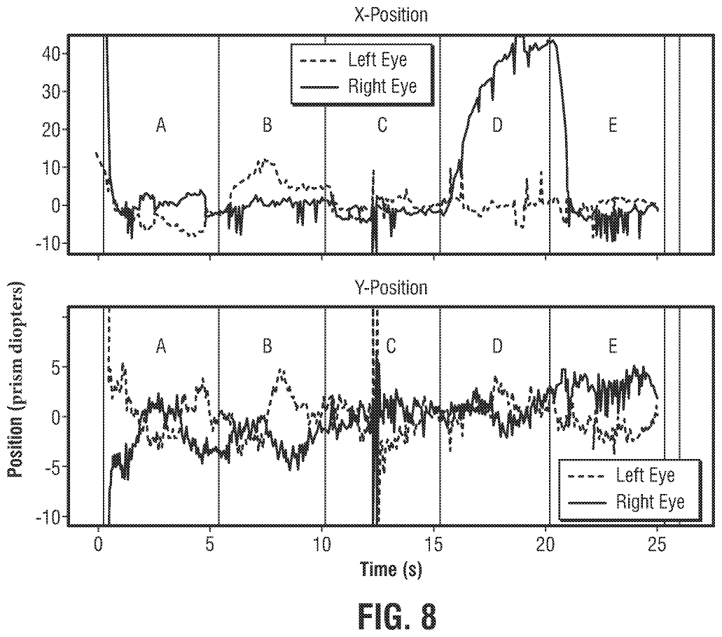

The processor may be configured to signal a further display, for example the display screen of the external computing device 9, to display the tracked positions of the left eye and the right eye during the cover/uncover testing. The positions may be displayed quantitatively as deviation or misalignment angles, qualitatively as a graphical depiction of the user's eyes that might exaggerate any such misalignment or deviation to make it easier for the ECP to notice, or a combination of both. FIG. 8 shows graphs of the tracked x-position (horizontal) and tracked y-position (vertical) of left and right eyes of a user who has been asked to fixate on an object being displayed in the VR headset 1, during cover/uncover testing. In this example, the sensitivity of the eye-tracking subsystem 8 is less than one degree (of eye orientation/position), and more particularly about 0.1 degree per pixel of the eye-tracking tracking subsystem. The example sequence of segments A-E shown in the figure are obtained by the processor signaling the left and right visible light displays as follows (while recording the tracked position of the left and right eyes when the user fixates on the object that they can see which is being displayed in the VR headset 1):
A) the object is displayed simultaneously in both the left display and in the right display for 5 seconds, to get fixation;
B) the object is displayed in only the right display for 5 seconds;
C) the object is displayed simultaneously in both the left display and in the right display for 5 seconds to regain fixation;
D) the object is displayed in only the left display for 5 seconds; and
E) the object is displayed simultaneously in both the left display and the right display for 5 seconds to regain fixation.

The eye tracking results shown in FIG. 8 indicate that the user may have exophoria in their right eye as evidenced by the sustained deviation of their right eye's x-position in segment D (while their right eye is “covered”). In this example, the exophoria detection threshold may be defined as about four degrees, but of course the thresholds for tropia and phoria detection may differ from that number.

To make an accurate assessment of a tropia, using the cover/uncover testing described above for the VR headset 1, the following calibration procedure may be performed. Note here that conventional calibration of a VR headset (to improve accuracy of its eye tracking) is performed with both eyes open. That means if a tropia is present, then the system registers the deviated or misaligned position of an eye as the “aligned” position, which in turn means that displaying the stimulus object in the left and right displays (for the cover/uncover testing) does not correspond exactly to a real-world stimulus. To address this problem, the processor can be configured to perform a calibration process for the VR headset 1 that in effect calibrates each eye both individually (sets the aligned position of each eye) and the left and right eyes together. The calibration process may be as follows:
instruct the user (wearing the VR headset 1) to fixate on an object that is being displayed by both the left and right displays (operation 20);
record a position of the left eye and a position of the right eye, as “combined calibration positions”, while the left and right visible light displays are simultaneously displaying the object that the user would see as a single object if their eyes were centered (no tropia)—(operation 22);
in operation record the tracked position of the right eye as an individual right eye calibration position when covering the left eye only (operation 24);
record the tracked position of the left eye as an individual left eye calibration position when covering the right eye only (operation 26); and
determine a deviation of the left eye based on a difference between i) the combined calibration position of the left eye and ii) the individual left eye calibration position (operation 28); and
determine a deviation of the right eye based on a difference between i) the combined calibration position of the right eye and ii) the individual right eye calibration position (operation 30).

When performing the above calibration process, if the user's eyes are aligned then the deviations should be “small” (below a threshold) assuming of course that the displayed stimulus object is in the center of the field of vision. But if the user has a tropia in their left eye, then there will be no movement of the eyes when the left eye is covered (see FIG. 5) but when the right eye is covered (see FIG. 6) the left eye will turn inward. So, for the example of FIGS. 4-6 (tropia in the left eye), the deviation determined in the above-described calibration process will be “large” or greater than the threshold. When the deviation is small, the processor records the individual or combined positions of the left and right eyes as calibration positions. In the case where the deviation is large, the processor records the individual (not the combined) calibration positions, and it may generate a notification that the current wearer's eyes are not aligned.

Another way to ensure accurate measurements, particularly in the case where the wearer has a tropia (because their eyes are not centered when both eyes are uncovered), is to track the position of an entirely of the eye, or the whole eye, not just the pupil position. If the whole eye is imaged, then the gaze direction of the pupil can be determined without calibration (e.g., using knowledge of the display distance, and the location of the pupil within the eye can determine gaze angle). In one aspect, to image the whole eye, the camera would need to capture more than the iris and sclera, and perhaps either up to and including the eyebrow or up to and including the margins the eye including left and right extremes and top and bottom eye lids. In either case, enough fiducials should be captured to know what the position of the pupil is relative to the head (or relative to the non-moving parts of the eye).

The position of the entire eye could be seen as an absolute position with the patient's head, instead of relative to a calibration position. The interpupillary distance, IPD, is measured as eye coordinates, and those are registered to the coordinates of the virtual stimulus object, and a virtual prism correction by deviation the stimulus in the eyes and seeing at which point the eyes do not move when they are covered in the cover/uncover test. This may be performed using an algorithm that converges on the solution, as compared to performing a linear or binary search. There is in this case no need to determine calibration positions of the eyes.

In one aspect, the eye tracking subsystem performs imaging of i) an entirety of the left eye to produce an imaged left eye, and ii) an entirety of the right eye to produce an imaged right eye, and the processor is configured to: shift coordinates of the object, resulting in shifted object coordinates, until the imaged right eye and the imaged left eye become aligned or centered; and convert the shifted object coordinates to an angular position or to a prism diopter measurement, of the left eye or of the right eye.

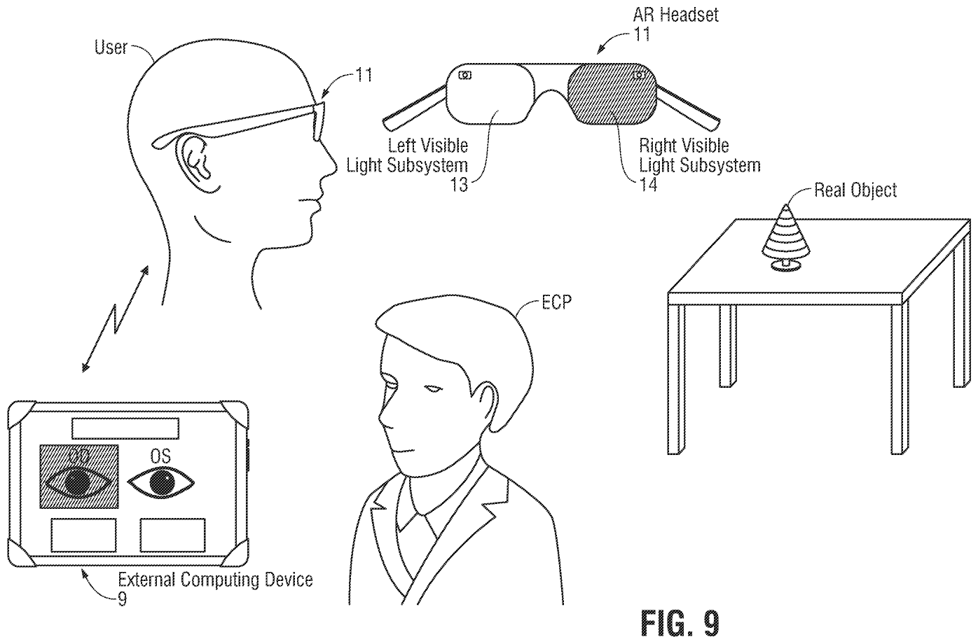

Turning now to FIG. 9, this is a diagram of an example, augmented reality, AR, headset-based system for cover/uncover testing. This system includes an AR headset 11 which may be composed of a left visible light subsystem positioned over a left eye of a user, and a right visible light subsystem positioned over a right eye of the user. Each respective visible light subsystem has a see-through mode of operation in which, with the AR headset 11 fitted to the user's head, the user can see a real object which is in an ambient scene, through the respective visible light subsystem. Additionally, each respective visible light subsystem has an occluded mode of operation in which the user cannot see the object that is in the ambient scene, through the respective visible light subsystem. As in the VR headset 1, the AR headset 11 also has a non-visible light-based eye tracking subsystem that can track a position of the left eye and a position of the right eye, e.g., an infrared pupil tracking subsystem.

For cover/uncover testing using this system, a processor is configured to, when the AR headset 11 has been fitted to the user's head, i) record the tracked position of the left eye while simultaneously i) signaling the left visible light subsystem into the occluded mode of operation, and ii) signaling the right visible light subsystem into the see-through mode of operation. This corresponds to only the left eye being “covered” while the user is asked to fixate on the real object. The recorded tracked position of the left eye while covered can be used to determine that the user has no tropia and no phoria in their left eye when the left eye shows no horizontal or vertical movement, as seen in FIG. 3. Similarly, the processor also records the tracked position of the right eye while simultaneously i) signaling the right visible light subsystem into the occluded mode of operation, and ii) signaling the left visible light subsystem into the see-through mode of operation. This corresponds to only the right eye being “covered” while the user is asked to fixate on the real object. The recorded tracked position of the right eye while covered can be used to determine that the user has no tropia and no phoria in their right eye when the right eye has no horizontal or vertical movement.

The rest of the cover/uncover testing scenarios (using the recorded tracked positions of the eyes) described above in connection with the VR headset 1 may also be made using the AR headset 11, except that the “covered” and “uncovered” states are achieved differently with the AR headset 11 (since there may not be any object that is displayed in the AR headset 11). Other aspects are similar to those described above for the VR headset based system, such as the processor being external to the AR headset 11, and the AR headset 11 comprises a wired or wireless communications network interface through which image data from the eye tracking subsystem is sent to the processor; or in another instance the tracked positions of the left eye and the right eye are sent to the external processor; and the processor is further configured to signal a further display screen, e.g., one that is integrated into the external computing device, to display the tracked positions of the left eye and the right eye as for example described above in connection with FIG. 8, or display the detected deviation of each eye.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. The description is thus to be regarded as illustrative instead of limiting.

1. A virtual reality, VR, headset-based system comprising:
a VR headset comprising
a left visible light display;
a left compartment to fit over a left eye of a user;
a right visible light display;
a right compartment to fit over a right eye of the user, wherein the left compartment and the right compartment are configured so that when the VR headset has been fitted over the user's eyes i) the user cannot see the right visible light display using only their left eye and ii) the user cannot see the left visible light display using only their right eye, and
a non-visible light-based eye tracking subsystem for tracking a position of the left eye and a position of the right eye; and
a processor configured to, when the VR headset has been fitted over the left eye and the right eye of the user,
i) signal the left visible light display and the right visible light display to display an object that the user sees as a single object while the left eye and the right eye are open,
ii) record the tracked position of the left eye while signaling the left visible light display to stop displaying the object while the object remains displayed in the right visible light display, and
iii) record the tracked position of the right eye while signaling the right visible light display to stop displaying the object while the object remains displayed in the left visible light display.

2. The system of claim 1 wherein the processor signals the left visible light display so the object disappears from the left visible light display but remains displayed as stationary in the right visible light display.

3. The system of claim 1 wherein the processor is external to the VR headset, and the VR headset comprises a wired or wireless communications network interface through which image data from the eye tracking subsystem is sent to the processor.

4. The system of claim 1 wherein the processor is external to the VR headset, and the VR headset comprises a wired or wireless communications network interface through which the tracked positions of the left eye and the right eye are sent to the processor.

5. The system of claim 1 wherein the processor is further configured to signal a further display to display the tracked positions of the left eye and the right eye.

6. The system of claim 1 wherein the eye tracking subsystem images an entirety of the left eye and an entirety of the right eye, and wherein the processor determines gaze angles of the left eye and the right eye based on:
knowledge of distance between the left eye and the left visible light display;
distance between the right eye and the right visible display, and
location of a left pupil within the left eye and a right pupil within the right eye, or interpupillary distance.

7. The system of claim 1 wherein the processor signaling the left display or the right display to stop displaying the object comprises
the processor signaling the left display or the right display to darken until the object disappears.

8. The system of claim 7 wherein the processor signaling the left display or the right display to stop displaying the object comprises
the processor signaling the left display or the right display to darken completely.

9. The system of claim 1 wherein the processor is further configured to, before ii) and iii) and while the user fixates on the object,
iv) record a combined calibration position of the left eye and a combined calibration position of the right eye while the left and right visible light displays are simultaneously displaying the object that the user would see as a single object, while their left and right eyes are both open simultaneously, if their eyes were centered.

10. The system of claim 1 wherein the processor is further configured to:
in ii), record the tracked position of the right eye as an individual right eye calibration position; and
in iii), record the tracked position of the left eye as an individual left eye calibration position; and
determine a deviation of the left eye or the right eye based on a difference between the combined calibrations positions in iv) and the individual right and left eye calibration positions.

11. The system of claim 10 wherein the processor is further configured to prepare data for displaying the deviation of the left eye or the right eye.

12. The system of claim 1 wherein the eye tracking subsystem for tracking the position of the left eye and the position of the right eye performs imaging of an entirety of the left eye and an entirety of the right eye, and the processor is configured to process said imaging to generate each of the tracked position of the left eye and the tracked position of the right eye as an absolute position with respect to the wearer's head, measure interpupillary distance, register virtual coordinates of the object to gaze coordinates, and define a virtual prism correction.

13. The system of claim 12 wherein the processor is configured to display the virtual prism correction as a measure of tropia of the user.

14. The system of claim 1 wherein the eye tracking subsystem performs imaging of i) an entirety of the left eye to produce an imaged left eye, and ii) an entirety of the right eye to produce an imaged right eye, and the processor is configured to:
shift coordinates of the object, resulting in shifted object coordinates, until the imaged right eye and the imaged left eye become aligned or centered; and
convert the shifted object coordinates to an angular position or to a prism diopter measurement, of the left eye or of the right eye.

15. The system of claim 14 wherein the processor is configured to display the prism diopter measurement as a measure of tropia of the user.

16. An augmented reality, AR, headset-based system, the system comprising:
an AR headset comprising
a left visible light subsystem positioned over a left eye of a user,
a right visible light subsystem positioned over a right eye of the user, wherein each respective visible light subsystem, of the left visible light subsystem and the right visible light subsystems, has i) a see-through mode of operation in which, with the AR headset fitted to a head of the user, the user can see an object that is in an ambient scene through the respective visible light subsystem, and ii) an occluded mode of operation in which the user cannot see the object that is in the ambient scene through the respective visible light subsystem,
a non-visible light-based eye tracking subsystem that can track a position of the left eye and a position of the right eye; and
a processor configured to, when the AR headset has been fitted to the head of the user,
i) record the tracked position of the left eye while simultaneously i) signaling the left visible light subsystem into the occluded mode of operation, and ii) signaling the right visible light subsystem into the see-through mode of operation, and
iii) record the tracked position of the right eye while simultaneously i) signaling the right visible light subsystem into the occluded mode of operation, and ii) signaling the left visible light subsystem into the see-through mode of operation.

17. The system of claim 16 wherein the processor is external to the headset, and the headset comprises a wired or wireless communications network interface through which image data from the eye tracking subsystem is sent to the processor.

18. The system of claim 16 wherein the processor is external to the headset, and the headset comprises a wired or wireless communications network interface through which the tracked positions of the left eye and the right eye are sent to the processor.

19. The system of claim 16 wherein the processor is further configured to signal a further display to display the tracked positions of the left eye and the right eye.

20. The system of claim 16 wherein the eye tracking subsystem is an infrared pupil tracking subsystem.
